(19) United States
(12) Patent Application Publication
(10) Pub. No.: US 2014/0193517 A1
(43) Pub. Date: Jul. 10, 2014

(54) METHOD OF DRUG REPOSITIONING

(71) Applicants: Pankaj Agarwal, King of Prussia, PA (US); Lun Yang, King of Prussia, PA (US)

(72) Inventors: Pankaj Agarwal, King of Prussia, PA (US); Lun Yang, King of Prussia, PA (US)

(73) Assignee: GlaxoSmithKline Intellectual Property Development Limited, Brentford, Middlesex (GB)

(21) Appl. No.: 14/239,564

(22) PCT Filed: Aug. 17, 2012
(86) PCT No.: PCT/US12/51257

§ 371 Date: Feb. 19, 2014

Related U.S. Application Data
(60) Provisional application No. 61/525,467, filed on Aug. 19, 2011.

(51) Int. Cl. G06F 19/00; G06N 5/04; G06N 99/00; A61K 45/06

(52) U.S. Cl. CPC G06F 19/3437; A61K 45/06; G06N 5/04; G06N 99/005
USPC 424/600; 514/17.6; 514/17.7; 514/21.9; 514/34; 514/179; 514/192; 514/338; 706/46; 706/12

(57) ABSTRACT

Methods are provided for selecting a new therapeutic indication for at least one first pharmaceutical comprising the steps of: generating a drug-side effect (SE) association for said at least one first pharmaceutical; generating a disease-side effect (SE) association for at least one disease or disorder and at least one second pharmaceutical intended for treatment of said at least one disease or disorder; determining an association strength between the drug-side effect (SE) association and the disease-side effect (SE) association; selecting said at least one disease or disorder as a new therapeutic indication for said at least one first pharmaceutical if said at least one first pharmaceutical induces at least one side effect which is the same as at least one side effect induced by at least one second pharmaceutical intended for treatment of said at least one disease or disorder.

### FIELD OF THE INVENTION

The invention relates to methods for selecting a therapeutic indication for a pharmaceutical as well as methods of treating various disease and disorders with a pharmaceutical.

### BACKGROUND OF THE INVENTION

Repositioning helps fully explore the indications of marketed drugs and clinical candidates (Ashburn, et al. Nat Rev Drug Discov 2004; 3(8):673-83); however, most successful stories of drug repositioning are based on serendipity but not systematic analysis (Sardan, et al. Brief Bioinform 2011). In silico methodologies have helped in mining the drug's off-target effects (Xie, et al. PLoS Comput Biol 2007; 3(11):e2173-7; Campillos, et al. Science 2008; 321(5886):263-6; Keiser, et al. Nature 2009; 462(7270):175-81; Yang, et al. PLoS Comput Biol 2009; 5(7):e1000441; and Luo, et al. Nucleic Acids Res 2011), off-system effects (such as, off-target related gene expression perturbation) (Suthram, et al. PLoS Comput Biol 2010; 6(2):e1000662; Yang, et al. PLoS Comput Biol 2011; 7(3):e1002016; Iorio, et al. Proc Natl Acad Sci USA 2010; 107(33):14621-6; and Hu, et al. PLoS One 2009; 4(8):e6536) and off-phenotypes (i.e. adverse drug reactions (Pouliot, et al. Clin Pharmacol Ther 2011; and Tatonetti, et al. Clin Pharmacol Ther 2011) or new indication) providing new hypotheses for in vitro assay (MacDonald, et al. Nat Chem Biol 2006; 2(6):329-37), animal model testing, or clinical trials to reposition the drug. The above strategies primarily focus on using preclinical information. Clinical therapeutic effects, however, are not always consistent with preclinical outcomes (Buchan, et al. Drug Discov Today 2011). Drug repositioning can help explore new indications of marketed drugs and clinical candidates.

Thus, methods are needed for identifying new or unsuspected indications for existing pharmaceuticals. Additionally, methods are needed for validating or invalidating a first therapeutic indication of a pharmaceutical as well as selecting at least a therapeutic agent for treatment or prevention of a disease and/or disorder.

### BRIEF DESCRIPTION OF THE DRAWINGS

FIG. 1. Constructing and visualization of the disease-SE associations. a) Confusion matrix of using SE priapism to predict Parkinson Disease. PD: Parkinson Disease; MCC: Matthews correlation coefficient; TP, FP, TN and FN stand for true positive, false positive, true negative and false negative respectively. Here one disease-SE pair could be represented by a confusion matrix. b) The overall layout of the disease-SE network. Diseases and SEs are in red and white circles respectively. The edge color and the width indicate the association strength measured by MCC. The neuropsychiatric, neoplasm, circulatory-system disease dominated clusters are highlighted in yellow, red and grey rectangles respectively. c) Neuropsychiatric disease-dominated cluster. The SE tardive dyskinesia and priapism is highlighted in orange. The MCC for PD priapism pair is 0.47 according to the confusion matrix in a) and is visualized as a yellow line. d) The performance of using priapism to predict whether or not a drug could treat a disease, which is measured using MCC, sensitivity and specificity.

FIG. 2. Sketch of StruSEf. a) Train the 566 SE models. For SEⱼ, the φⱼ^{pos} (+) and φⱼ^{neg} (−) were recruited from 888 SIDER molecules. b) The diseaseᵢ-moleculeₖ association (Θᵢₖ) was calculated as the dot product value of the disease-SE association vector (DS) and SE-molecule association vector (SM). The binary SE-molecule (SM) association was calculated from QSAR models. The width of the colored lines indicates the weights of the disease-SE associations. As an example, Θᵢ₂ is more than Θᵢ₁ as the association of side effect j in green to disease i is stronger.

FIG. 3. Predict drugs' repositioning potential for hypertension via DRoSEf. a) The distribution of the 0 score for the positive (red) and negative (blue) set for hypertension. The molecules with high 0 score in negative set (red square bracket) were chosen as the candidates for treating hypertension. b) The ROC curve of using 0 score to predict hypertension. The AUC is 0.74. c) Predicted relationships of the top molecules with the 12 SEs and the association of these SEs with the hypertension. The binary association among molecules and SEs is in grey lines. The association strength between SE and disease is reflected on the color and the width of the edge. Postural hypotension is highlighted as the SE explicitly linked to hypertension.

### SUMMARY OF THE INVENTION

In one embodiment, methods are provided for selecting a new therapeutic indication for at least one first pharmaceutical comprising the steps of:
generating a drug-side effect (SE) association for said at least one first pharmaceutical;
generating a disease-side effect (SE) association for at least one disease or disorder and at least one second pharmaceutical intended for treatment of said at least one disease or disorder;
determining an association strength between the drug-side effect (SE) association and the disease-side effect (SE) association;
selecting said at least one disease or disorder as a new therapeutic indication for said at least one first pharmaceutical if said at least one first pharmaceutical induces at least one side effect which is the same as at least one side effect induced by at least one second pharmaceutical intended for treatment of said at least one disease or disorder.

### DETAILED DESCRIPTION

As used herein “pharmaceutical” means any active ingredient capable of treating or preventing at least one disease, trait and/or phenotype. The pharmaceutical compositions of the invention are prepared using techniques and methods known to those skilled in the art. Some of the methods commonly used in the art are described in Remington's Pharmaceutical Sciences (Mack Publishing Company).

As used herein “druggable” means a characteristic that allows a compound or composition to be developed into a drug. For example, a druggable compound or composition could have at least one of the following characteristics: capable of being formulated for administration to a mammal, capable of reaching its target once administered to a mammal, and/or capable of effecting at least one target. Similarly, the term “biopharmable” refers to large molecule such as, but not limited to, proteins, antibodies, antibody fragments, domain antibodies, single chain antibodies, bispecific antibodies, and any combination or variations thereof, aptamers, fusion proteins, synthetic polypeptides, recombinant polypeptides, vaccines, DNA therapies, and/or RNAi, that can be administered to a mammal.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein “reposition” and “repositioning” and grammatical variations thereof refers to a disease, trait and/or phenotype for which a pharmaceutical may have a use beyond the first disease, trait and/or phenotype for which the pharmaceutical had identified activity.

As used herein the term “amplification” and grammatical variations thereof refers to the presence of one or more extra gene copies in a chromosome complement. In certain embodiments a gene encoding a Ras protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

As used herein “overexpressed” and “overexpression” of a protein or polypeptide and grammatical variations thereof means that a given cell produces an increased number of a certain protein relative to a normal cell of the same type. By way of example, a protein may be overexpressed by diseased cell relative to a normal cell. Additionally, a mutant protein may be overexpressed compared to wild type protein in a cell. As is understood in the art, expression levels of a polypeptide in a cell can be normalized to a housekeeping gene such as actin. In some instances, a certain polypeptide may be underexpressed in a cell compared with a normal or standard cell.

As used herein “drug-side effect (SE) association” refers to an association of at least one side effect with at least one pharmaceutical.

As used herein “disease-side effect (SE) association” refers to a association of at least one but maybe more than one side effect that may be induced by at least one pharmaceutical or a class of drugs intended for treatment of a certain disease or disorder.

In one embodiment, methods are provided for selecting a new therapeutic indication for at least one first pharmaceutical comprising the steps of:
generating a drug-side effect (SE) association for said at least one first pharmaceutical;
generating a disease-side effect (SE) association for at least one disease or disorder and at least one second pharmaceutical intended for treatment of said at least one disease or disorder;
determining an association strength between the drug-side effect (SE) association and the disease-side effect (SE) association;
selecting said at least one disease or disorder as a new therapeutic indication for said at least one first pharmaceutical if said at least one first pharmaceutical induces at least one side effect which is the same as at least one side effect induced by at least one second pharmaceutical intended for treatment of said at least one disease or disorder.

In one aspect the invention includes counting the number of drugs inducing or not inducing a SE when treating or not treating a disease, and generating a confusion matrix.

In one aspect the drug-side effect (SE) association is determined by the pharmaceutical label of said first pharmaceutical. In one aspect the drug-side effect (SE) association is determined by the SIDER database.

In one aspect the association strength is determined using Matthew correlation coefficient (MCC). In one aspect the association strength of is determined using sensitivity (sn). In one aspect the association strength of (c) is determined using specificity (sp).

In another embodiment, methods are provided for treating a mammal in need of treatment with at least one of the diseases listed as New Indication in Table 1 with the corresponding pharmaceutical listed as Drug of Table 1. In one aspect the mammal is a human.

TABLE 1

| Drug | New Indication |
|---|---|
| acebutolol | Cardiomyopathy, Dilated |
| acebutolol | Hypercholesterolemia |
| allopurinol | Carotid Artery Diseases |
| allopurinol | Depressive Disorder |
| alprazolam | Depressive Disorder, Major |
| alprazolam | Depression, Postpartum |
| alprazolam | Obsessive-Compulsive Disorder |
| amiodarone | Carotid Artery Diseases |
| anastrozole | Lung Neoplasms |
| atorvastatin | Obsessive-Compulsive Disorder |
| atorvastatin | Depression, Postpartum |
| atorvastatin | Parkinson Disease |
| azathioprine | Lung Neoplasms |
| bezafibrate | Obsessive-Compulsive Disorder |
| bortezomib | Depression |
| bortezomib | Depressive Disorder, Major |
| bupropion | Cardiomyopathy, Hypertrophic |
| bupropion | Parkinson Disease |
| busulfan | Diabetes Mellitus |
| cabergoline | Depression |
| cabergoline | Depression, Postpartum |
| cabergoline | Depressive Disorder, Major |
| cabergoline | Lung Neoplasms |
| cabergoline | Ovarian Neoplasms |
| candesartan | Carotid Artery Diseases |
| candesartan | Depressive Disorder |
| capecitabine | Carotid Artery Diseases |
| capecitabine | Hypercholesterolemia |
| capecitabine | Hyperlipidemias |
| carbamazepine | Depression, Postpartum |
| carbamazepine | Obsessive-Compulsive Disorder |
| carvedilol | Lung Neoplasms |
| ceftriaxone | Depressive Disorder |
| celecoxib | Osteoporosis |
| celecoxib | Osteoporosis, Postmenopausal |
| cerivastatin | Cardiomyopathy, Hypertrophic |
| cerivastatin | Stroke |
| chlorambucil | Depression |
| chlorpromazine | Obsessive-Compulsive Disorder |
| cilazapril | Depressive Disorder |
| cilazapril | Obsessive-Compulsive Disorder |
| cisapride | Anxiety Disorders |
| cisapride | Depression, Postpartum |
| cisapride | Depressive Disorder, Major |
| cisapride | Obsessive-Compulsive Disorder |
| clomipramine | Carotid Artery Diseases |
| clomipramine | Epilepsy |
| clomipramine | Hypercholesterolemia |
| clomipramine | Hyperlipidemias |
| clomipramine | Osteoporosis |
| clomipramine | Osteoporosis, Postmenopausal |
| clomipramine | Parkinson Disease |
| clonidine | Depression, Postpartum |
| clopidogrel | Lung Neoplasms |
| clozapine | Cardiomyopathy, Dilated |
| clozapine | Cardiomyopathy, Hypertrophic |
| clozapine | Depression, Postpartum |
| clozapine | Depressive Disorder, Major |
| clozapine | Epilepsy |
| cyclophosphamide | Carotid Artery Diseases |
| cyclosporine | Depression, Postpartum |
| cyclosporine | Depressive Disorder |
| cyclosporine | Depressive Disorder, Major |
| cyclosporine | Obsessive-Compulsive Disorder |
| desipramine | Depression, Postpartum |
| desipramine | Cardiomyopathy, Hypertrophic |
| dexamethasone | Depression, Postpartum |
| dipyridamole | Depressive Disorder |
| dipyridamole | Obsessive-Compulsive Disorder |
| donepezil | Depression, Postpartum |
| donepezil | Obsessive-Compulsive Disorder |
| doxazosin | Obsessive-Compulsive Disorder |
| doxazosin | Carotid Artery Diseases |
| doxazosin | Depressive Disorder |
| doxazosin | Parkinson Disease |
| doxazosin | Schizophrenia |
| doxepin | Parkinson Disease |
| doxepin | Schizophrenia |
| doxorubicin | Epilepsy |
| doxorubicin | Obsessive-Compulsive Disorder |
| droperidol | Cardiomyopathy, Hypertrophic |
| duloxetine | Obsessive-Compulsive Disorder |
| duloxetine | Depression, Postpartum |
| duloxetine | Cardiomyopathy, Hypertrophic |
| efavirenz | Depression, Postpartum |
| efavirenz | Carotid Artery Diseases |
| efavirenz | Depressive Disorder, Major |
| eletriptan | Obsessive-Compulsive Disorder |
| eletriptan | Depressive Disorder |
| eletriptan | Carotid Artery Diseases |
| estradiol | Cardiomyopathy, Hypertrophic |
| exemestane | Ovarian Neoplasms |
| famotidine | Depressive Disorder, Major |
| famotidine | Lung Neoplasms |
| fenofibrate | Obsessive-Compulsive Disorder |
| flecainide | Epilepsy |
| fludarabine | Pain |
| fluoxetine | Carotid Artery Diseases |
| fluoxetine | Osteoporosis |
| fluoxetine | Osteoporosis, Postmenopausal |
| fluvastatin | Cardiomyopathy, Dilated |
| fluvastatin | Cardiomyopathy, Hypertrophic |
| fluvoxamine | Parkinson Disease |
| fluvoxamine | Cardiomyopathy, Hypertrophic |
| fluvoxamine | Carotid Artery Diseases |
| fluvoxamine | Diabetes Mellitus |
| formoterol | Depressive Disorder |
| formoterol | Obsessive-Compulsive Disorder |
| fosinopril | Depressive Disorder |
| fosinopril | Obsessive-Compulsive Disorder |
| gabapentin | Carotid Artery Diseases |
| gabapentin | Depression, Postpartum |
| gabapentin | Hypercholesterolemia |
| gabapentin | Hyperlipidemias |
| gadodiamide | Depressive Disorder, Major |
| ganciclovir | Hypercholesterolemia |
| ganciclovir | Cardiomyopathy, Dilated |
| ganciclovir | Cardiomyopathy, Hypertrophic |
| ganciclovir | Carotid Artery Diseases |
| ganciclovir | Hyperlipidemias |
| glatiramer acetate | Obsessive-Compulsive Disorder |
| glatiramer acetate | Depressive Disorder |
| glatiramer acetate | Depression, Postpartum |
| glatiramer acetate | Breast Neoplasms |
| glatiramer acetate | Carotid Artery Diseases |
| glatiramer acetate | Depression |
| glatiramer acetate | Depressive Disorder, Major |
| glatiramer acetate | Epilepsy |
| glatiramer acetate | Parkinson Disease |
| glatiramer acetate | Schizophrenia |
| glibenclamide | Depression |
| glimepiride | Depression |
| glipizide | Depression |
| granisetron | Depressive Disorder, Major |
| heparin | Obsessive-Compulsive Disorder |
| heparin | Parkinson Disease |
| hydrochlorothiazide | Anxiety Disorders |
| hydrochlorothiazide | Depressive Disorder, Major |
| hydrochlorothiazide | Obsessive-Compulsive Disorder |
| hydroxyurea | Carotid Artery Diseases |
| ifosfamide | Depression |
| imatinib | Cardiomyopathy, Hypertrophic |
| imatinib | Depression |
| imatinib | Depression, Postpartum |
| imatinib | Depressive Disorder |
| imipramine | Depression, Postpartum |
| ketorolac | Obsessive-Compulsive Disorder |
| lamivudine | Carotid Artery Diseases |
| lamotrigine | Obsessive-Compulsive Disorder |
| lamotrigine | Depression, Postpartum |
| lamotrigine | Osteoporosis |
| lamotrigine | Osteoporosis, Postmenopausal |
| lansoprazole | Depressive Disorder |
| lansoprazole | Obsessive-Compulsive Disorder |
| lansoprazole | Depression |
| lansoprazole | Depression, Postpartum |
| latanoprost | Osteoporosis |
| latanoprost | Osteoporosis, Postmenopausal |
| leflunomide | Lung Neoplasms |
| leflunomide | Obsessive-Compulsive Disorder |
| lenalidomide | Cardiomyopathy, Hypertrophic |
| lenalidomide | Breast Neoplasms |
| lenalidomide | Lung Neoplasms |
| levetiracetam | Depressive Disorder, Major |
| levonorgestrel | Anxiety Disorders |
| levonorgestrel | Depression |
| levonorgestrel | Depression, Postpartum |
| levonorgestrel | Depressive Disorder |
| levonorgestrel | Depressive Disorder, Major |
| lidocaine | Depression, Postpartum |
| lidocaine | Depressive Disorder |
| lidocaine | Depressive Disorder, Major |
| lidocaine | Obsessive-Compulsive Disorder |
| lithium | Depression, Postpartum |
| lovastatin | Cardiomyopathy, Hypertrophic |
| loxapine | Depression, Postpartum |
| loxapine | Depressive Disorder, Major |
| loxapine | Obsessive-Compulsive Disorder |
| memantine | Depression, Postpartum |
| memantine | Obsessive-Compulsive Disorder |
| methamphetamine | Cardiomyopathy, Hypertrophic |
| methyldopa | Anxiety Disorders |
| methyldopa | Depression, Postpartum |
| methyldopa | Depressive Disorder, Major |
| methyldopa | Obsessive-Compulsive Disorder |
| methylphenidate | Cardiomyopathy, Hypertrophic |
| mexiletine | Cardiomyopathy, Hypertrophic |
| mexiletine | Hypercholesterolemia |
| mexiletine | Stroke |
| mifepristone | Anxiety Disorders |
| mifepristone | Depression, Postpartum |
| mifepristone | Obsessive-Compulsive Disorder |
| modafinil | Depression, Postpartum |
| modafinil | Obsessive-Compulsive Disorder |
| morphine | Epilepsy |
| naproxen | Obsessive-Compulsive Disorder |
| naproxen | Depressive Disorder |
| naproxen | Osteoporosis |
| naproxen | Osteoporosis, Postmenopausal |
| nefazodone | Depression, Postpartum |
| nelfinavir | Carotid Artery Diseases |
| nevirapine | Breast Neoplasms |
| nevirapine | Lung Neoplasms |
| nevirapine | Ovarian Neoplasms |
| olanzapine | Depression, Postpartum |
| olanzapine | Carotid Artery Diseases |
| olanzapine | Epilepsy |
| omeprazole | Carotid Artery Diseases |
| omeprazole | Depressive Disorder |
| omeprazole | Hypercholesterolemia |
| omeprazole | Hyperlipidemias |
| omeprazole | Obsessive-Compulsive Disorder |
| ondansetron | Depressive Disorder, Major |
| oseltamivir | Depression |
| oseltamivir | Depression, Postpartum |
| oseltamivir | Depressive Disorder, Major |
| oxaliplatin | Obsessive-Compulsive Disorder |
| oxcarbazepine | Obsessive-Compulsive Disorder |
| oxcarbazepine | Depression, Postpartum |
| oxcarbazepine | Depressive Disorder, Major |
| oxcarbazepine | Parkinson Disease |
| oxycodone | Depression |
| paclitaxel | Depressive Disorder, Major |
| pamidronate | Lung Neoplasms |
| paroxetine | Carotid Artery Diseases |
| pemoline | Epilepsy |
| penicillin g | Obsessive-Compulsive Disorder |
| penicillin g | Schizophrenia |
| pergolide | Obsessive-Compulsive Disorder |
| pergolide | Depression, Postpartum |
| pergolide | Schizophrenia |
| perindopril | Cardiomyopathy, Hypertrophic |
| phenytoin | Carotid Artery Diseases |
| phenytoin | Depression, Postpartum |
| phenytoin | Obsessive-Compulsive Disorder |
| pindolol | Hypercholesterolemia |
| posaconazole | Lung Neoplasms |
| pramipexole | Obsessive-Compulsive Disorder |
| pramipexole | Depression, Postpartum |
| pramipexole | Breast Neoplasms |
| pramipexole | Carotid Artery Diseases |
| pramipexole | Epilepsy |
| pravastatin | Cardiomyopathy, Dilated |
| pravastatin | Cardiomyopathy, Hypertrophic |
| prednisolone | Obsessive-Compulsive Disorder |
| prednisolone | Anxiety Disorders |
| prednisolone | Depression, Postpartum |
| prednisone | Anxiety Disorders |
| prednisone | Carotid Artery Diseases |
| prednisone | Depression, Postpartum |
| prednisone | Depressive Disorder, Major |
| prednisone | Obsessive-Compulsive Disorder |
| procarbazine | Depressive Disorder |
| procarbazine | Epilepsy |
| propoxyphene | Carotid Artery Diseases |
| propylthiouracil | Lung Neoplasms |
| quetiapine | Depression, Postpartum |
| rabeprazole | Depressive Disorder |
| rabeprazole | Depression, Postpartum |
| rabeprazole | Carotid Artery Diseases |
| rabeprazole | Depression |
| rabeprazole | Depressive Disorder, Major |
| rabeprazole | Hypercholesterolemia |
| rabeprazole | Hyperlipidemias |
| rabeprazole | Lung Neoplasms |
| rabeprazole | Obsessive-Compulsive Disorder |
| reserpine | Osteoporosis |
| reserpine | Osteoporosis, Postmenopausal |
| ribavirin | Obsessive-Compulsive Disorder |
| risperidone | Depression, Postpartum |
| ritonavir | Depressive Disorder |
| ritonavir | Depressive Disorder, Major |
| ritonavir | Lung Neoplasms |
| ritonavir | Obsessive-Compulsive Disorder |
| ritonavir | Osteoporosis, Postmenopausal |
| rivastigmine | Obsessive-Compulsive Disorder |
| rivastigmine | Depression, Postpartum |
| rivastigmine | Depressive Disorder, Major |
| rivastigmine | Epilepsy |
| rofecoxib | Carotid Artery Diseases |
| rosuvastatin | Cardiomyopathy, Dilated |
| rosuvastatin | Cardiomyopathy, Hypertrophic |
| saquinavir | Carotid Artery Diseases |
| saquinavir | Depression |
| saquinavir | Depression, Postpartum |
| saquinavir | Depressive Disorder |
| sibutramine | Obsessive-Compulsive Disorder |
| sildenafil | Obsessive-Compulsive Disorder |
| sildenafil | Depression, Postpartum |
| simvastatin | Cardiomyopathy, Hypertrophic |
| sotalol | Hypercholesterolemia |
| sulfasalazine | Cardiomyopathy, Hypertrophic |
| sumatriptan | Depression, Postpartum |
| sumatriptan | Depressive Disorder, Major |
| sumatriptan | Carotid Artery Diseases |
| sumatriptan | Epilepsy |
| sumatriptan | Parkinson Disease |
| tacrolimus | Epilepsy |
| tadalafil | Obsessive-Compulsive Disorder |
| tadalafil | Parkinson Disease |
| tadalafil | Schizophrenia |
| tamoxifen | Obsessive-Compulsive Disorder |
| tamoxifen | Parkinson Disease |
| tegaserod | Depressive Disorder |
| tegaserod | Obsessive-Compulsive Disorder |
| telmisartan | Obsessive-Compulsive Disorder |
| temozolomide | Carotid Artery Diseases |
| temozolomide | Depression, Postpartum |
| temozolomide | Depressive Disorder |
| temozolomide | Depressive Disorder, Major |
| temozolomide | Obsessive-Compulsive Disorder |
| testosterone | Obsessive-Compulsive Disorder |
| testosterone | Cardiomyopathy, Dilated |
| testosterone | Cardiomyopathy, Hypertrophic |
| thalidomide | Depression |
| thalidomide | Depressive Disorder |
| thalidomide | Depressive Disorder, Major |
| thalidomide | Anxiety Disorders |
| thalidomide | Cardiomyopathy, Hypertrophic |
| thalidomide | Depression, Postpartum |
| thioridazine | Obsessive-Compulsive Disorder |
| thioridazine | Depression, Postpartum |
| thioridazine | Depressive Disorder, Major |
| tiagabine | Depression, Postpartum |
| tiagabine | Obsessive-Compulsive Disorder |
| timolol | Cardiomyopathy, Hypertrophic |
| timolol | Cardiomyopathy, Dilated |
| timolol | Hypercholesterolemia |
| timolol | Obsessive-Compulsive Disorder |
| tolbutamide | Depression |
| tolcapone | Depression |
| tolcapone | Depression, Postpartum |
| tolcapone | Depressive Disorder, Major |
| tolcapone | Obsessive-Compulsive Disorder |
| topiramate | Depression, Postpartum |
| topiramate | Cardiomyopathy, Hypertrophic |
| tramadol | Depressive Disorder |
| tramadol | Carotid Artery Diseases |
| tramadol | Depression, Postpartum |
| tramadol | Depressive Disorder, Major |
| trazodone | Depression, Postpartum |
| triamcinolone | Anxiety Disorders |
| triamcinolone | Carotid Artery Diseases |
| triamcinolone | Depression, Postpartum |
| triamcinolone | Depressive Disorder |
| triamcinolone | Depressive Disorder, Major |
| triamcinolone | Obsessive-Compulsive Disorder |
| trifluoperazine | Depressive Disorder, Major |
| trifluoperazine | Obsessive-Compulsive Disorder |
| trifluoperazine | Depression |
| trifluoperazine | Depression, Postpartum |
| trifluoperazine | Depressive Disorder |
| trimipramine | Anxiety Disorders |
| trovafloxacin | Depression, Postpartum |
| trovafloxacin | Depressive Disorder |
| trovafloxacin | Depressive Disorder, Major |
| trovafloxacin | Obsessive-Compulsive Disorder |
| venlafaxine | Osteoporosis |
| venlafaxine | Osteoporosis, Postmenopausal |
| venlafaxine | Parkinson Disease |
| venlafaxine | Carotid Artery Diseases |
| verapamil | Depressive Disorder, Major |
| verapamil | Depression, Postpartum |
| verapamil | Obsessive-Compulsive Disorder |
| vincristine | Cardiomyopathy, Hypertrophic |
| vincristine | Epilepsy |
| vincristine | Obsessive-Compulsive Disorder |
| vinorelbine | Depression |
| warfarin | Obsessive-Compulsive Disorder |
| warfarin | Parkinson Disease |
| zanamivir | Depression |
| zanamivir | Depression, Postpartum |
| zanamivir | Depressive Disorder, Major |
| ziprasidone | Obsessive-Compulsive Disorder |
| ziprasidone | Depression, Postpartum |
| ziprasidone | Breast Neoplasms |
| ziprasidone | Carotid Artery Diseases |
| ziprasidone | Epilepsy |
| zolpidem | Depression, Postpartum |
| zolpidem | Obsessive-Compulsive Disorder |
| zolpidem | Osteoporosis |
| zolpidem | Osteoporosis, Postmenopausal |
| zonisamide | Obsessive-Compulsive Disorder |

The invention is further described by the following non-limiting examples.

### EXAMPLES

### Example 1

### Drug Re-Positioning Based on Side Effects

Here we show that the clinical side-effects (SEs) produced by a drug provide a human phenotypic profile for the drug, and this profile can suggest additional indications. The rationale behind this methodology is compelling in that both therapeutic effects and side effects are behavioral or physiological changes in response to the drug, and may be associated with each other via known or unknown mechanism-of-action (MOA). We extracted 3,175 SE-disease relationships by combining the SE-drug relationships from drug label and the drug-disease relationships from PharmGKB. Many relationships provide explicit repurposing hypotheses, such as drugs causing SE hypoglycemia are potential candidates for diabetes, and a different dose or formulation may possibly produce a clinically beneficial effect in at least a sub population that shows the side effect. Based on these 3,175 relationships, an indication prediction model was constructed. The model was subsequently tested on 4,200 clinical candidates across 101 major human diseases. 36% of the disease models achieved an AUC higher than 0.7, including depression, anxiety disorders, stomach neoplasms, non-small-cell lung carcinoma, lymphoma, leukemia and type II diabetes. The MOA for each SE-disease association was also investigated to rationally interpret the prediction result. This study suggests that clinical pharmacologists should pay closer attention to the SEs observed in clinical trials not just to evaluate the potentially harmful side effects, but to also rationally explore the repositioning potential based on this “clinical human phenotypic assay”.

Repositioning helps fully explore the indications of marketed drugs and clinical candidates (Ashburn, et al. Nat Rev Drug Discov 2004; 3(8):673-83); however, most successful stories of drug repositioning are based on serendipity but not systematic analysis (Sardan, et al. Brief Bioinform 2011). In silico methodologies have helped in mining the drug's off-target effects (Xie, et al. PLoS Comput Biol 2007; 3(11):e2173-7; Campillos, et al. Science 2008; 321(5886):263-6; Keiser, et al. Nature 2009; 462(7270):175-81; Yang, et al. PLoS Comput Biol 2009; 5(7):e1000441; and Luo, et al. Nucleic Acids Res 2011), off-system effects (such as, off-target related gene expression perturbation) (Suthram, et al. PLoS Comput Biol 2010; 6(2):e1000662; Yang, et al. PLoS Comput Biol 2011; 7(3):e1002016; Iorio, et al. Proc Natl Acad Sci USA 2010; 107(33):14621-6; and Hu, et al. PLoS One 2009; 4(8):e6536) and off-phenotypes (i.e. adverse drug reactions (Pouliot, et al. Clin Pharmacol Ther 2011; and Tatonetti, et al. Clin Pharmacol Ther 2011) or new indication) providing new hypotheses for in vitro assay (MacDonald, et al. Nat Chem Biol 2006; 2(6):329-37), animal model testing, or clinical trials to reposition the drug. The above strategies primarily focus on using preclinical information. Clinical therapeutic effects, however, are not always consistent with preclinical outcomes (Buchan, et al. Drug Discov Today 2011). Drug repositioning can help explore new indications of marketed drugs and clinical candidates.

Recently, a systematic analysis pointed out that phenotypic screening exceeded target-based approaches in discovering first-in-class small-molecule drugs (Swinney, et al. Nat Rev Drug Discov 2011; 10(7):507-19). Clinical phenotypic information comes from actual patient data, which mimics a phenotypic “screen” of drug effects on human, and can directly help rational drug repositioning. For example, one study tried to suggest drug's new indications based on existing therapeutic effect (Chiang, et al. Clin Pharmacol Ther 2009; 86(5):507-10). In our study, however, we utilize the rich information of the clinical side-effects (SEs), which usually regarded as unwanted effects of the drugs, to suggest new indications for a drug. For instance, hypotension is an unfavorable SE of many drugs. However, they may act as candidate anti-hypertension drug if we utilize this SE via controlling the dosing, improve the formulation and choosing the sub-population etc.

The rationale for this strategy is that SEs and indications are both measurable behavioral or physiologic changes in response to the treatment, and if drugs treating the same disease share the same SE, there might be some underline mechanism-of-actions (MOAs) linking this disease and the SE and this SE could serve as the phenotypic “marker” of the therapeutic effect of this disease. Furthermore, both therapeutic and side effects are observations on human subjects, but not animal models, so there is less translational issue. This is to suggest the understanding more about the conditions and the extent to which the SE was produced may warrant additional experiments as to MOA, and perhaps eventual repositioning. In this study, we systematically examined the connections between SEs and indications, and quantitatively measured the power of using these connections to predict new indications.

The methodology of Drug Repositioning based on the Side-Effectome (DRoSEf) is discussed in this study. The basic hypothesis is that if the SEs associated with a drug D are also induced by many of the drugs treating disease X, then drug D should be evaluated as a candidate for treating X. We constructed a database of disease-SE associations. Clinical pharmacologists, who observe a SE in their clinical trial can query the database for diseases for which there are drugs that have the same side effect. This would suggest alternative indications for the drug in the clinical trial. The biologists can also investigate the underlying MOA for the relationship between SEs and disease so as to better understand the pathogenesis and the therapeutic process of the disease. Using this approach, we predicted new indications for marketed drugs and 4,200 candidate drugs that were in the clinical trial, with the prediction performance quantitatively measured.

### Results

### Identification of the Disease-Side Effect Associations

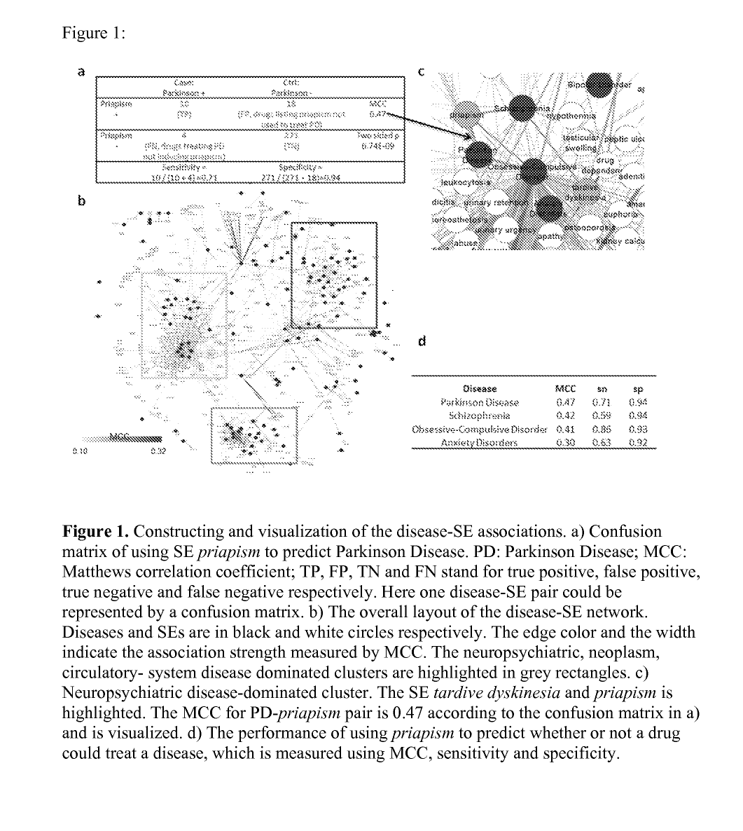

Both disease-drug associations and drug-SE associations are required to infer disease-SE associations. We extracted the indications of drugs from PharmGKB to provide the disease-drug associations (Altman, et al. Nat Genet 2007; 39(4):426). There are multiple resources for SE information. The SEs printed on drug label, however, provide consistent and reliable data as these are identified from large clinical trials, and the drug label is approved and standardized by regulatory agencies. The SIDER database (Kuhn, et al. Mol Syst Biol 2010; 6:343), which had been used to predict drug off-target (Campillos, et al. Science 2008; 321(5886):263-6), provides drug-SE relationships extracted from drug labels for 888 approved drugs using text-mining (Yang, et al. Bioinformatics 2009; 25(17):2244-50 and Agarwal, et al. Nat Rev Drug Discov 2009; 8(11):865-78). The relationships among drugs and SEs were extracted from this database. We just used the binary fact of the SE's presence on the drug label. We then inferred disease-SE associations by counting the number of the drugs inducing or not inducing a SE when treating or not treating a disease, generating confusion matrix as shown in FIG. 1a. The association strength of a disease-SE pair is measured using multiple criteria, including the Matthews correlation coefficient (MCC), sensitivity (sn) and specificity (sp). We computed 84,680 confusion matrices for each pair of 145 diseases and 584 SEs. 3,175 (3.75%) of these associations were considered possibly informative (using multiple criteria as described in Methods).

We investigated a few of the 3,175 associations to understand what these associations implied and how they can be used to suggest new indications. Some of the associations have the explicit explanation based on the current knowledge of the MOA (Table 2). 1) The SE positive ANA indicates the presence of autoimmune antibodies and appears to be associated with stroke. It is the SE shared by drugs treating stroke, mainly ticlopidine and several angiotensin-converting enzyme (ACE) inhibitors. Stroke itself, is associated with severe immune suppression (Vogelgesang, et al. J Neuroimmunol 2011; 231(1-2):105-10). Thus, conceivably drugs that are associated with increasing immune response in terms of positive ANA may help stroke patients, though of course an autoimmune response is not desirable. Overall, 50% of the drugs treating stroke were listed this SE whereas only 2% drug not listed as treating stroke were listed it. These 2% drugs were called false positive drugs (Table 2). Several statins are associated with positive ANA, but not indicated for stroke. A meta-analysis of 120,000 patients across 42 trials showed that statin therapy provides protection for all-cause mortality and nonhemorrhagic strokes (O'Regan, et al. Am J Med 2008; 121(1):24-33). Ramipril, which associates with positive ANA, also shows a 32% risk reduction for stroke [11909785]. The immune related mechanism of action (MOA) in stroke has only been recognized recently (Vogelgesang, et al.), however, based on identification of the stroke positive ANA association by DRoSEf, it is likely that the immune related SEs of these drugs could help suggest their potential use for stroke regardless this MOA was recognized. 2) Cytomegalovirus infection is a sign of a weakened immune system (Dechanet, et al. J Infect Dis 1999; 179(1):1-8). Drugs that reduce immune response are often used to prevent transplant rejection, thus drugs that list increased cytomegalovirus (CMV) infections as a SE may be good candidates for treating transplant patients. Methotrexate, an antineoplastic drug, lists CMV infections as a SE. It has been indicated for preventing transplant rejection [8956122]. 3) DRoSEf suggests that drugs that list porphyria as SE may act as antidiabetics. There are significant negative association between porphyria and diabetes (Andersson, et al. J Intern Med 1999; 245(2):193-7 and Yalouris, et al. Br Med J (Clin Res Ed) 1987; 295(6608):1237-8), with the MOA unknown. For example, in a study of 328 Swedish patients with porphyria, the 16 patients that developed diabetes all had their porphyria symptoms resolved. Valproic acid is a mood-stabilizing drug that lists porphyria. A recent study found it effective in lowering blood glucose levels in both Wfs1 knockout mice (Terasmaa, et al. J Physiol Biochem 2011). Pyrazinamide is an anti-tuberculosis agent that lists porphyria. Interestingly, tuberculosis was found to be correlated with diabetes (Cantalice, et al. J Bras Pneumol 2007; 33(6):691-8 and Nijland, et al. Clin Infect Dis 2006; 43(7):848-54). In mice, naproxen may be a valuable tool to delay or prevent the development of type II diabetes from a pre-diabetic condition (Kendig, et al. Biochem Pharmacol 2008; 76(2):216-24). Estradiol was also found to have antidiabetic effect (Kumar, et al. Endocrinology 2011). In a double-blinded, randomized placebo controlled clinical trial on women with type II diabetes, oral estradiol significantly decreased fasting glucose [19339356]. 4) Drugs that list delusions as a side effect may help with depression. Cabergoline, an ergot derivative that causes delusions, is a dopamine agonist that has an antidepressant-like property (Chiba, et al. Psychopharmacology (Berl) 2010; 211(3):291-301). The dopamine receptor agonist pergolide has shown antidepressant effects in Parkinson patients (Picillo, et al. Parkinsonism Relat Disord 2009; 15 Suppl 4:S81-S84 and Quan, et al. Neuroscience 2011; 182:88-97). 5) Hyperacusis is a medical condition associated with hypersensitivity to certain frequency ranges of sounds. Phenytoin is a known anticonvulsant with hyperacusis as a listed side effect, and DRoSEf suggests a potential utility for treating depression. In fact, a small clinical trial found equivalent therapeutic effects between phenytoin and fluoxetine in treating depression [15889944], the latter drug being the first line antidepressant agent. Modafinil is a drug for narcolepsy and is also potentially effective in combination with fluoxetine to treat depression (Abolfazli, et al. Depress Anxiety 2011; 28(4):297-302). 6) Constitutional symptoms are a listed SE for many antineoplasm drug. An anti-HIV drug nevirapine also lists constitutional symptoms as a SE. Nevirapine has previously been suggested as a treatment for human hormone-refractory prostate carcinoma (Landriscina, et al. Prostate 2009; 69(7):744-54) and other neoplasms (Landriscina, et al. Int J Cancer 2008; 122(12):2842-50).

TABLE 2
Some of the associations from the disease—SE network

| Disease | | | | | | | False Positive | |
|---|---|---|---|---|---|---|---|---|
| Class | Disease | Side Effect | MCC | sn | sp | p value | Drugs^{a} | Reference |
| Circulation | Stroke | positive ANA | 0.46 | 0.47 | 0.98 | 1.77E−15 | statins, ramipril | (39), (40), |
| System | | | | | | | | (41) |
| Other | Transplantation | Cytomegalovir | 0.75 | 0.75 | 0.99 | 3.48E−06 | methotrexate | [8956122] |
| | | us infection | | | | | | |
| Metabolite | Diabetes | porphyria | 0.44 | 0.50 | 0.98 | 8.78E−06 | valproic acid, | (28), |
| disease | Mellitus | | | | | | pyrazinamide, | (29;30), |
| | | | | | | | naproxen, | (31), (32) |
| | | | | | | | estradiol | |
| Psychiatric | Depressive | delusions | 0.46 | 1.00 | 0.91 | 1.13E−08 | cabergoline, | (33), (35), |
| disease | Disorder | | | | | | memantine, | (34) |
| | | | | | | | pergolide | |
| | Depressive | hyperacusis | 0.55 | 0.88 | 0.96 | 8.96E−09 | phenytoin, | (42), (36) |
| | Disorder | | | | | | modafinil | |
| Neoplasms | Neoplasms | constitutional | 0.50 | 0.56 | 0.94 | 2.64E−18 | nevirapine | (37;38) |
| | | symptoms | | | | | | |

^{a}Drugs not listed treating disease (2^{nd} column) but listed the SE (3^{rd} column).

In fact, 27% of the “false positive” drugs-disease association suggested by DRoSEf have at least one clinical trial article listed in PubMed. However, not all 3,175 associations have an obvious MOA explanation based on current knowledge. Based on these 3,175 associations, we further built Naïve Bayes models to predict the 145 indication endpoints using their associated SEs as the features. The average AUCs of 10-fold cross validations for each of the 145 disease were calculated using Weka (Mark Hall EFGHBPPRIHW. The WEKA Data Mining Software: An Update; SIGKDD Explorations. 11[1]. Jan, 1, 2009), 92% of the which were above the 0.8.

### Visualization of the Disease-SE Associations

Based on these 3,175 associations, a disease-SE network was constructed (FIG. 1b). Diseases that share similar SEs tend to be cluster with each other. The diseases are grouped into three clusters dominated by neuropsychiatric diseases (FIG. 1c), circulatory system diseases, and neoplasms as visualized using Cytoscape, et al. Bioinformatics 2011; 27(3):431-2). The neuropsychiatric disease-dominated cluster (FIG. 1c) shares SEs, such as tardive dyskinesia, an involuntary movement SE associated with long term dosing or high doses of antipsychotics [12801428]. Other SEs, such as priapism, a painful medical condition in which the erect penis or clitoris does not return to flaccid state [16698365], is also shared by four neuropsychiatric diseases (FIG. 1d). 86% of the drugs that treat obsessive-compulsive disorder (OCD) list priapism as a side effect; whereas only 7% of the drugs not reported to treat OCD list this SE. The connection between priapism and OCD could provide two hypotheses for repositioning. First, the 7% of the drugs that are not indicated for OCD, but list priapism as a side effect may be potential treatments for OCD, similar to the suggestions based on the examples in Table 2. Of course one would have to consider central nervous system penetration of the drug and other biological rationale as well. Secondly, although priapism is regarded as an unfavorable SE during the therapies of some neuropsychiatric diseases (Compton M T, Miller A H. J Clin Psychiatry 2001; 62(5):362-6), it might present a repositioning opportunity with perhaps a different formulation or dose of such drugs for sexual dysfunction therapies.

Moreover, among the 7% of the drugs that have not been indicated for OCD in PharmGKB but list priapism as a SE, some of them have been reported to treat OCD in literature. For example, ziprasidone has been used as the coadjuvant treatment in resistant OCD treatment (Iglesias, et al. Actas Esp Psiquiatr 2006; 34(4):277-9); quetiapine was reported to be effective in treating OCD (Alexander, et al. Aust N Z J Psychiatry 2009; 43(12):1185; Vulink, et al. J Clin Psychiatry 2009; 70(7):1001-8; and Savas, et al. Clin Drug Investig 2008; 28(7):439-42); there is a case report of oxcarbazepine's therapeutic effect in OCD (McMeekin H. J S C Med Assoc 2002; 98(8):316-20); the symptom of OCD could be decreased after olanzapine treatment (van, et al. J Clin Psychopharmacol 2008; 28(2):214-8); a trend toward an non-obsessive response was seen under nefazodone treatment (Nelson EC. Ann Clin Psychiatry 1994; 6(4):249-53); strong reduction of the OCD could be observed after using clozapine (Peters B, de H L. Prog Neuropsychopharmacol Biol Psychiatry 2009; 33(8):1576-7). Sildenafil was also among the 7% of the drugs that cause ‘priapism’. Given the association of OCD and priapism, and the central nervous system penetration of sildenafil (Schultheiss, et al. World J Urol 2001; 19(1):46-50), the drug could be considered for OCD. A possible MOA is that nitric oxide modulates the neurotransmitters implicated in OCD (Umathe, et al. Nitric Oxide 2009; 21(2):140-7), and the inhibition of PDE5 protein by sildenafil may lead to a sustained release of nitric oxide (Ghofrani, et al. Nat Rev Drug Discov 2006; 5(8):689-702).

### DRoSEf Based on Small Molecular Structure

The above analysis requires knowing the SEs from a drug's label before we predict new indications. We also wanted to investigate whether we could predict SEs based on compound structure and then predict new indications based on those SEs. We hypothesized that such a prediction “chain” would provide mechanistic explanations of the compound's new indication based on the disease-SE association and the structural information of the compound. To present a quantitative framework of DRoSEf's performance, we recruited all small molecule candidates or marketed drugs from Genego® MetaBase (Ekins, et al. Expert Opin Drug Metab Toxicol 2005; 1(2):303-24). This provided a data source of 4,200 additional molecules in addition to the 888 SIDER drugs. These 4,200 molecules are indicated for at least one of 101 diseases from the 145 disease set. MetaBase also uses MeSH disease terms, thus making comparisons to the MeSH indications from PharmGKB straightforward.

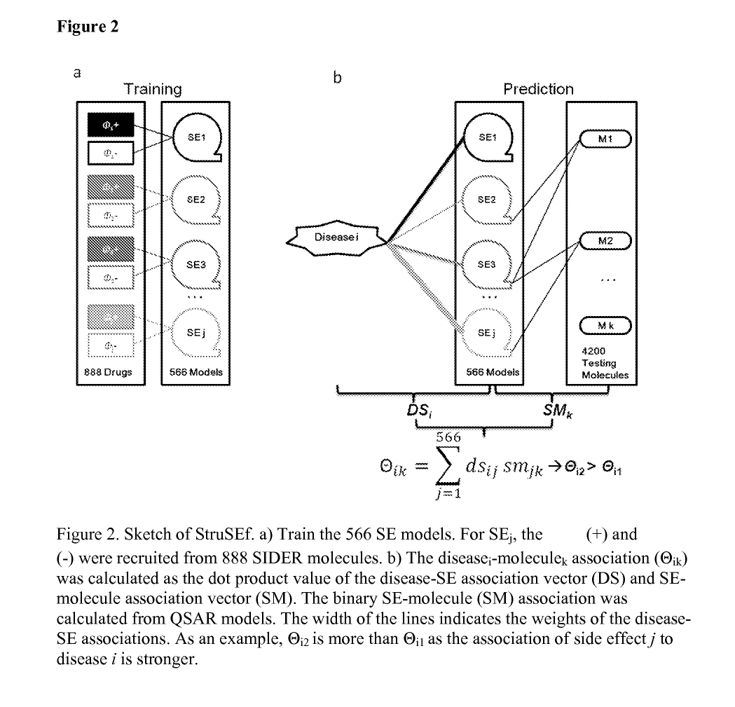

DRoSEf requires the side-effect profile for each molecule to predict new indications. However, such information is difficult to obtain for most of the 4,200 molecules because they are generally clinical candidates without FDA approved drug labels, and have little or no SE published from their clinical trials in a standardized way. Quantitative structure-activity relationship (QSAR) models have been used to predict target binding of the ligand (Nidhi, et al. J Chem Inf Model 2006; 46(3):1124-33). We hypothesized that QSAR models could also be used to predict SEs, and the use of the predicted SEs as an intermediate towards predicting a disease indication would help understand the underpinnings of the disease indication, but also not lower the quality of the disease indication prediction. We are attempting to go from drug Structure to Side Effect and then to a disease indication (StruSEf). For side effect j (SEⱼ), we recruited the positive set (φⱼ^{pos}, drugs causing SEⱼ) and the negative set (φⱼ^{neg}, drugs not reported to induce SEⱼ) from the 888 SIDER drug set (FIG. 2a). For 566 of the 584 SEs, we successfully trained QSAR models (18 of them failed). Then we used these 566 QSAR models to predict the SEs of 4,200 molecules from MetaBase. Each molecule k could then be represented as a binary vector (SMₖ) of size 566 with position j being one if and only if this drug would be predicted to have side effect j. Each disease i was also independently associated with a vector (DS) of 566 SEs as computed earlier (and shown in the network in FIG. 1) based on the data from SIDER. To evaluate if molecule k could be used to treat disease i, we then sum up the 566 products for each of the elements in disease-SE (DS) and the SE-molecule (SMₖ) vector. A higher score suggests that the molecule has been predicted to induce more SEs with higher association strength with the disease (FIG. 2b). For each disease i, we use the dot product value (Θᵢ) as the metric to calculate the area under curve (AUC) of the ROC.

The ROC curves of the prediction performance for 101 disease endpoints were generated. Some of the disease endpoints had only a few positive drugs from the MetaBase set, and their AUC value might not accurately reflect the true performance. We, therefore, focus on the diseases that have more than 30 compounds with that specific indication in MetaBase. Table 3 lists the diseases with AUC greater than 0.70. The AUCs for neuropsychiatric diseases are higher than neoplasms and other disease endpoints, which may be due to a higher number of the SEs (and thus a better characterized side effect profile) for these diseases. We then evaluated the extent of the structure similarity information contributed to these performances. In fact, when we do not use SE information at all and rely only on chemical structure, only 18% of the 101 disease endpoints achieve AUCs above 0.7, while 36% of disease endpoints had AUCs higher than 0.7 from StruSEf. Moreover, 74% of endpoints achieved higher AUC in StruSEf than using chemical structural information alone. Only 22% of the variance in the AUCs of StruSEf was explained by “chemical structural only” across the 101 endpoints. This again indicates that the side effect intermediate is adding value to the prediction.

TABLE 3
StruSEf prediction results for several disease endpoints based on predicting side
effects from structure, and then using side effects to predict an indication.

| | | # of drugs | # of SE | |
|---|---|---|---|---|
| | | with this | features | |
| | | indication in | associated | |
| Disease category | Disease | clinical trial | with disease | AUC |
| Neuropsychiatric | Depression | 72 | 87 | 0.82 |
| | Depressive Disorder | 42 | 204 | 0.82 |
| | Schizophrenia | 77 | 55 | 0.81 |
| | Depressive Disorder, Major | 48 | 170 | 0.81 |
| | Anxiety Disorders | 144 | 186 | 0.71 |
| Neoplasms | Stomach Neoplasms | 49 | 4 | 0.77 |
| | Carcinoma, Non-Small-Cell Lung | 73 | 10 | 0.76 |
| | Lung Neoplasms | 59 | 30 | 0.74 |
| | Neoplasms | 347 | 42 | 0.74 |
| | Lymphoma | 28 | 4 | 0.72 |
| | Leukemia, Myeloid, Acute | 30 | 20 | 0.71 |
| | Head and Neck Neoplasms | 33 | 7 | 0.70 |
| Others | Hypertension | 203 | 12 | 0.74 |
| | Diabetes Mellitus, Type 2 | 112 | 8 | 0.71 |

### Case Study of StruSEf for Predicting Molecules Treating Hypertension

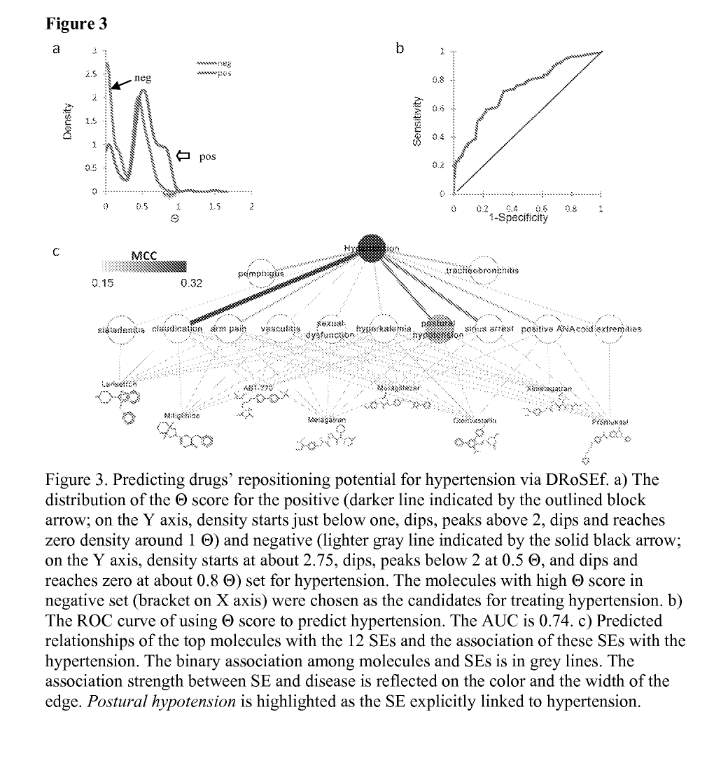

MetaBase includes 203 molecules indicated for hypertension. However, there are molecules other than the 203 above that have not yet been reported to treat hypertension that achieved a relatively high Θ score based on SEs (corresponding to the rightmost part of the blue line in FIG. 3a). There are 12 side effects linked to hypertension. Structure of some of the molecules with the highest Θ and their predicted relationships with the 12 hypertension-associated SEs are visualized in FIG. 3c, many of which are physiologically linked to hypertension (Supporting Information 1). Noteworthy, Postural hypotension is the low blood pressure that occurs after suddenly standing or sitting up. Drugs causing this SE should possibly be considered and evaluated for treating hypertension provided the effect can be controlled with formulation and dosing. Nine of the top 10 molecules predicted to effect hypertension from MetaBase are also predicted to induce postural hypotension, which is perhaps a relevant clinical phenotypic screen for hypertension and adds direct evidence for potential repositioning (FIG. 3c).

Among the top investigational molecules in FIG. 3c, glenvastatin is originally indicated for hyperlipidemias. Studies have documented the effect of statins on blood pressure [12147931, 12473855]. Melagatran and ximelagatran are the thrombin inhibitors. Thrombin signaling was proved to be involved in the vascular response to hypertension [9168786]. Muraglitazar is an agonist of PPARα and PPARγ. PPARα stimulation exerts a lowering effect in blood pressure [19857485]; whereas the SEs of PPARγ agonists usually include lowering of blood pressure [20069049]. ABT-770 is a metalloproteinase inhibitor, and the metalloproteinase was reported to regulate blood pressure [19850948].

Blonanserin acts as the antagonist of 5-HT2 receptor. A study demonstrated that the increase in blood pressure is due to a stimulation of postjunctional 5-HT2 receptors [3368008].

Some hypertension specific SEs are associated with MOA of various classes of antihypertensive drugs. Thus, we hypothesize that other drugs that are not known as treatments for hypertension but show those side effects might well be in part acting through that specific MOA. For example, pemphigus is reported to be induced by angiotensin-converting enzyme (ACE) inhibitors (Ong, et al. Australas J Dermatol 2000; 41(4):242-6.), and cold extremities could be induced by antihypertensives especially by 13-adrenergic blockers (Feleke, et al. Acta Med Scand 1983; 213(5):381-5.). In the prediction results from StruSEf, we also found that ACE inhibitors are significant enriched in the drugs predicted as pemphigus positive (Fisher's exact p=1.4E-3); whereas β-adrenergic blockers have significantly higher frequency in drugs predicted as cold extremities positive (p=0.02).

### Discussion

This study proposes the systematic and rational drug repositioning based on SEs (DRoSEf), and demonstrated its applicability via prediction of the repositioning potential for 4,200 (candidate) drugs across 101 diseases. Based on the fact that the methodology could recall the known indications for the drug molecules, we suggested the unknown indications for both marketed drugs (DRoSEf) and the clinical candidates (StruSEf). Afterwards, these suggestions were evaluated via mining the proof-of-concepts from literature. The concept of the DRoSEf suggests that the clinical pharmacologists pay closer attention to the SEs observed in clinical trials, and thus explore additional indications for their drugs based on understanding the connections between SE and the therapeutic effect of the drug. The examples raised in this study are only for demonstrating the principle of this methodology, but may not necessary be effective or practical for real repurposing practice. Furthermore, lots of factors should be considered during the practical aspects of this methodology, such as the unmet medical need for the disease, the fraction of the population showing the side effect, the CNS penetration of the molecule and whether the therapeutic effect is significant enough in comparison to current treatments. Moreover the previous therapeutic effect could now become a potential side effect as well, and will need to be carefully considered in the risk benefit profile. But, hopefully, in a few cases this could all be managed via choosing a suitable formulation, dose, and the sub population.

The SEs could be used to predict the targets [18621671]. However, the basic principle of DRoSEf is to mimic “phenotypic assay” rather than the target based assay to screen compounds for a disease indication, though DRoSEf itself could suggest target, such as ACE and β-adrenergic receptor in the case study for hypertension. It has been reported that phenotypic screening exceeded that of target-based approaches to the discovery of first-in-class drugs [21701501]. DRoSEf leverages “assays” with direct human phenotypes. Our study demonstrated that the phenotypic features from human work well on suggesting new indication, which may even outperform assays running on in vitro models or on animal models that face translational challenges. Its application relies on the MOA of the association of the SEs to the disease, although many of these MOAs are unknown or complicated. In this study, we did not consider the absolute frequency of the SEs or the relative frequency or significance compared to placebo. In SIDER, only 37.9% of the drug-SE pairs have frequency information associated with them, thus to maximize the amount of drugs covered, SEs with higher frequencies like nausea and vomiting are usually described in detail and written in the drug label. However, the frequencies for most of the informative SEs are unknown. Some of the SEs are regarded to be rare, but are still implicated in the pathogenesis of a particular disease. In fact, they might expose an extreme phenotype of a known or unknown MOA. For example, porphyria is a rare inherited disease [11117426]. Patients with this inherited disease show a decrease in the risk of porphyria on becoming diabetic (26;27). This may suggest why antidiabetic drugs are usually reported to worsen porphyria, but this may only affect people with an inherited genetic mutation for porphyria, and this subset of population may act as the “model” for screening the anti-diabetes drugs, with porphyria as the screening endpoint. Thus, a drug that increases porphyria in this sub population with the mutation may well be a good diabetes drug in a different larger population. So the off-phenotype of a drug on a sub population might suggest its use for a broader population. Besides mimic a human phenotypic “screening” to help “fishing” the positive candidates for repositioning, DRoSEf may also suggest the unrecognized disease pathogenesis, such as studying the porphyria may lead to better understanding of the diabetes.

A limitation of StruSEf is the number (888) of drugs that have available side effects. The models and accuracy would improve if we were able to obtain side effects on a larger number of drugs. Moreover, predictions of indications for 4200 MetaBase drugs would also be better if we had some side effect information from their early stage clinical trials rather than relying on just their structures. Even if we had to rely on structures for preclinical molecules, it would help if the structure based side effect models were trained on more than the 888 drugs from SIDER. Constructing a larger database of disease-SE associations via mining the drug labels and additional literature should improve accuracy. On the other hand, the prediction performance could also be an underestimate. Molecules that have not yet been reported to treat a disease may well be capable of treating that disease, and in many cases (the false positive drugs as shown in Table 2) clinical trials have already shown a positive effect. These molecules are regarded as false positives currently. which decreases the computed AUC value. However, even with this imperfect SE information and potentially underestimated prediction performance, 36% of the disease endpoints achieved AUCs higher than 0.7, which is generally higher than the disease prediction performance using the QSAR model alone.

Using multiple SEs features to predict the disease endpoint could also improve sensitivity over individual features. Although there are explicit individual disease-SE associations, not all of them have sufficient prediction power. For instance, not all drugs treating anemia have been annotated with polycythemia in SIDER, thus the sensitivity of this feature is limited. The inclusion of multiple features could enhance sensitivity. If a true positive is not recalled by an individual feature, it still has a chance to be recalled by other features. In the case of hypertension, the drug candidate “pranlukast” cannot be recalled through the feature pemphigus, but can be recalled via “cold extremities” (FIG. 3c). Thus better sensitivity could be achieved if we had more SEs annotations or other phenotypic terms from drug label. The emphasis on the sensitivity, however, may affect the specificity. To avoid this problem, all the SEs chosen for the prediction have high specificity (sp>0.75, see Methods). Furthermore, the false positives could be excluded through further testing on in vivo, in vitro and animal models.

Inspecting the numerous examples of SE that certainly correspond to and may have even led to clinical trials for new indications, it is obvious that the clinical side effect observations provide powerful human clinical data that is already widely used implicitly by clinicians to repurpose drugs. DRoSEf systemizes this process, provides numerous predictions based on the underlying MOA of the SE in disease's pathogenesis, and benefits from the fact that side effects are human phenotypic data obviating translation issues.

### Methods

### Construct the Disease-Side Effect Associations

The disease-SE associations were computed based on the disease-drug association and drug-SE association, which were extracted from PharmGKB (18) and SIDER (19) databases respectively. PharmGKB uses MeSH term to describe diseases (Hansen, et al. Clin Pharmacol Ther 2009; 86(2):183-9). For side effects from SIDER, we only use them as present or absent in association with a drug, and do not consider their frequencies explicitly, as only 37.9% of the drugs had side effect frequencies associated with them. Let true positive (tpᵢⱼ) be the number of drugs listing that are indicated for disease i and list j as a SE; false positives (fpᵢⱼ) be the number of drugs that are not indicated for disease i and list SE j; true negatives (tnᵢⱼ) be the number of drugs that are not indicated for disease i and do not list SE j; false negatives (fnᵢⱼ) be the number of drugs that are indicated for i and do not list SE j. We calculated the sensitivity (snᵢⱼ), specificity (spᵢⱼ) and Matthews correlation coefficient or MCC (mccᵢⱼ) of using SE j to predict disease i using the standard formulas below:

$${sn}_{ij} = {tp}_{ij} / ( {tp}_{ij} + {fn}_{ij} ) , \\ {sp}_{ij} = {tn}_{ij} / ( {fp}_{ij} + {tn}_{ij} ) , \\ {mcc}_{ij} = \frac{( {tp}_{ij} {tn}_{ij} - {fp}_{ij} {fn}_{ij} )}{\sqrt{( {tp}_{ij} + {fp}_{ij} ) ( {tp}_{ij} + {fn}_{ij} ) ( {fn}_{ij} + {tn}_{ij} ) ( {fp}_{ij} + {tp}_{ij} )}} .$$

For binary variables, the MCC is the equivalent of a Pearson correlation coefficient. The two-sided Fisher's exact pᵢⱼ value was also calculated. A disease-SE association was considered to be non-informative, if (pᵢⱼ>0.05|mccᵢⱼ<0.15|spᵢⱼ<0.75|tpᵢⱼ<2). This threshold provided 3,175 informative associations including 145 MeSH disease phenotypes and 584 SEs. The associations in Table 2 was selected based on the following criteria: the MCC is among the top 150 of all 3175 associations; the tpᵢⱼ>3; the associations between the disease and the SE have an explanation according to the knowledge of the authors. In FIG. 1, to enhance the visibility of the network layout, the disease-SE relationships were not visualized if (pᵢⱼ>0.05|mccᵢⱼ<0.20|spᵢⱼ<0.80|tp<2).

### Train the Prediction Models in StruSEf

We calculated several structural descriptors (log P, molecular weight, number of hydrogen bond donors and acceptors, number of rotatable bonds and SCFP6 fingerprint) for 888 SIDER drugs. We tried to train 584 SE models with multiple Laplacian-modified Bayesian method (Nidhi, et al. J Chem Inf Model 2006; 46(3):1124-33) using the features above. 566 SE models were successfully trained.

### Predict the Disease Endpoints for Genego Molecule Set Based on SEs

We evaluated 5,534 clinical candidates or marketed drugs from Genego MetaBase (by January 2011). We considered only molecules that included SMILES strings, and further listed a disease indication matching at least one of the 145 from the SIDER set, and we excluded molecules that were duplicates from the SIDER drug set. This left us with 4,200 small molecules for an independent test set. These molecules were assigned at least one of the 101 disease MeSH term that match the 145 MeSH diseases.

The endpoint of our prediction is whether or not the compound should be considered for a clinical trial for treating disease i just based on side effect information. For each disease i, we computed its side-effectome profile vector from the SIDER data,

DSᵢ=[dsᵢ₁,dsᵢ₂, . . . ,dsᵢⱼ],jε[1,566],iε[1,101],

where dsᵢⱼ quantifies the association of disease i and SE j. The vectors were generated using seven different metrics, i.e.,

dsᵢⱼε{bᵢⱼ,mccᵢⱼ,mccᵢⱼ⁴,seᵢⱼ,seᵢⱼ⁴,spᵢⱼ,spᵢⱼ⁴},

where bᵢⱼ=0 if (pᵢⱼ>0.05|mccᵢⱼ<0.15|spᵢⱼ<0.75|aᵢⱼ<2), else, bᵢⱼ=1. We used the exponent four in an effort to enhance the signal of the high mcc, se or sp.

For each molecule k without known SEs, we estimated its side-effectome profile vector SMₖ by computing it using each of the 566 pre-trained SE models,

SMₖ=[sm₁ₖ,sm₂ₖ, . . . ,smⱼₖ],jε[1,566],kε[1,4200],

where smⱼₖ=1 if the molecule k was predicted as possibly causing SE j, else smⱼₖ=0. We calculate the association Θᵢₖ between disease i and molecule k as the dot product of the two vectors,

$${Θ}_{ik} = 〈 {DS}_{ij} , {SM}_{jk} 〉 = {\sum }_{j = 1}^{566} {ds}_{ij} {sm}_{jk}$$

We computed Θᵢₖ using each of the seven metrics, and for each metric we further computed an AUC for each of the 101 endpoints. The metrics seᵢⱼ⁴ performed best among all metrics in terms of the mean AUC across all 101 disease endpoints. Thus, the AUC value is based on the seᵢⱼ⁴ metrics.

### REFERENCE LIST FOR EXAMPLE 1

(1) Ashburn T T, Thor K B. Drug repositioning: identifying and developing new uses for existing drugs. Nat Rev Drug Discov 2004; 3(8):673-83.
(2) Sardana D, Zhu C, Zhang M, Gudivada R C, Yang L, Jegga A G. Drug repositioning for orphan diseases. Brief Bioinform 2011.
(3) Xie L, Wang J, Bourne P E. In silico elucidation of the molecular mechanism defining the adverse effect of selective estrogen receptor modulators. PLoS Comput Biol 2007; 3(11):e217.
(4) Campillos M, Kuhn M, Gavin A C, Jensen U, Bork P. Drug target identification using side-effect similarity. Science 2008; 321(5886):263-6.
(5) Keiser M J, Setola V, Irwin J J, Laggner C, Abbas A I, Hufeisen S J et al. Predicting new molecular targets for known drugs. Nature 2009; 462(7270):175-81.
(6) Yang L, Chen J, He L. Harvesting candidate genes responsible for serious adverse drug reactions from a chemical-protein interactome. PLoS Comput Biol 2009; 5(7):e1000441.
(7) Luo H, Chen J, Shi L, Mikailov M, Zhu H, Wang K et al. DRAR-CPI: a server for identifying drug repositioning potential and adverse drug reactions via the chemical-protein interactome. Nucleic Acids Res 2011.
(8) Suthram S, Dudley J T, Chiang A P, Chen R, Hastie T J, Butte A J. Network-based elucidation of human disease similarities reveals common functional modules enriched for pluripotent drug targets. PLoS Comput Biol 2010; 6(2):e1000662.
(9) Yang L, Wang K, Chen J, Jegga A G, Luo H, Shi L et al. Exploring Off-Targets and Off-Systems for Adverse Drug Reactions via Chemical-Protein Interactome—Clozapine-Induced Agranulocytosis as a Case Study. PLoS Comput Biol 2011; 7(3):e1002016.
(10) Iorio F, Bosotti R, Scacheri E, Belcastro V, Mithbaokar P, Ferriero R et al. Discovery of drug mode of action and drug repositioning from transcriptional responses. Proc Natl Acad Sci USA 2010; 107(33):14621-6.
(11) Hu G, Agarwal P. Human disease-drug network based on genomic expression profiles. PLoS One 2009; 4(8):e6536.
(12) Pouliot Y, Chiang A P, Butte A J. Predicting Adverse Drug Reactions Using Publicly Available PubChem BioAssay Data. Clin Pharmacol Ther 2011.
(13) Tatonetti N P, Denny J C, Murphy S N, Fernald G H, Krishnan G, Castro V et al. Detecting Drug Interactions From Adverse-Event Reports: Interaction Between Paroxetine and Pravastatin Increases Blood Glucose Levels. Clin Pharmacol Ther 2011.
(14) MacDonald M L, Lamerdin J, Owens S, Keon B H, Bilter G K, Shang Z et al. Identifying off-target effects and hidden phenotypes of drugs in human cells. Nat Chem Biol 2006; 2(6):329-37.
(15) Buchan N S, Rajpal D K, Webster Y, Alatorre C, Gudivada R C, Zheng C et al. The role of translational bioinformatics in drug discovery. Drug Discov Today 2011.
(16) Swinney D C, Anthony J. How were new medicines discovered? Nat Rev Drug Discov 2011; 10(7):507-19.
(17) Chiang A P, Butte A J. Systematic evaluation of drug-disease relationships to identify leads for novel drug uses. Clin Pharmacol Ther 2009; 86(5):507-10.
(18) Altman R B. PharmGKB: a logical home for knowledge relating genotype to drug response phenotype. Nat Genet 2007; 39(4):426.
(19) Kuhn M, Campillos M, Letunic I, Jensen L J, Bork P. A side effect resource to capture phenotypic effects of drugs. Mol Syst Biol 2010; 6:343.
(20) Campillos M, Kuhn M, Gavin A C, Jensen L J, Bork P. Drug target identification using side-effect similarity. Science 2008; 321(5886):263-6.
(21) Yang L, Xu L, He L. A CitationRank algorithm inheriting Google technology designed to highlight genes responsible for serious adverse drug reaction. Bioinformatics 2009; 25(17):2244-50.
(22) Agarwal P, Searls D B. Can literature analysis identify innovation drivers in drug discovery? Nat Rev Drug Discov 2009; 8(11):865-78.
(23) Vogelgesang A, Dressel A. Immunological consequences of ischemic stroke: immunosuppression and autoimmunity. J Neuroimmunol 2011; 231(1-2):105-10.
(24) O'Regan C, Wu P, Arora P, Perri D, Mills E J. Statin therapy in stroke prevention: a meta-analysis involving 121,000 patients. Am J Med 2008; 121(1):24-33.
(25) Dechanet J, Merville P, Berge F, Bone-Mane G, Taupin J L, Michel P et al. Major expansion of gammadelta T lymphocytes following cytomegalovirus infection in kidney allograft recipients. J Infect Dis 1999; 179(1):1-8.
(26) Andersson C, Bylesjo I, Lithner F. Effects of diabetes mellitus on patients with acute intermittent porphyria. J Intern Med 1999; 245(2):193-7.
(27) Yalouris A G, Raptis S A. Effect of diabetes on porphyric attacks. Br Med J (Clin Res Ed) 1987; 295(6608):1237-8.
(28) Terasmaa A, Soomets U, Oflijan J, Punapart M, Hansen M, Matto V et al. Wfs1 mutation makes mice sensitive to insulin-like effect of acute valproic acid and resistant to streptozocin. J Physiol Biochem 2011.
(29) Cantalice Filho J P, Boia M N, Sant Anna C C. [Analysis of the treatment of pulmonary tuberculosis in elderly patients at a university hospital in Rio de Janeiro, Brazil]. J Bras Pneumol 2007; 33(6):691-8.
(30) Nijland H M, Ruslami R, Stalenhoef J E, Nelwan E J, Alisjahbana B, Nelwan R H et al. Exposure to rifampicin is strongly reduced in patients with tuberculosis and type 2 diabetes. Clin Infect Dis 2006; 43(7):848-54.
(31) Kendig E L, Schneider S N, Clegg D J, Genter M B, Shertzer H G. Over-the-counter analgesics normalize blood glucose and body composition in mice fed a high fat diet. Biochem Pharmacol 2008; 76(2):216-24.
(32) Kumar R, Balhuizen A, Amisten S, Lundquist I, Salehi A. Insulinotropic and Antidiabetic Effects of 17 {beta}-Estradiol and the GPR30 Agonist G-1 on Human Pancreatic Islets. Endocrinology 2011.
(33) Chiba S, Numakawa T, Ninomiya M, Yoon H S, Kunugi H. Cabergoline, a dopamine receptor agonist, has an antidepressant-like property and enhances brain-derived neurotrophic factor signaling. Psychopharmacology (Berl) 2010; 211(3):291-301.
(34) Picillo M, Rocco M, Barone P. Dopamine receptor agonists and depression in Parkinson's disease. Parkinsonism Relat Disord 2009; 15 Suppl 4:S81-S84.
(35) Quan M N, Zhang N, Wang Y Y, Zhang T, Yang Z. Possible antidepressant effects and mechanisms of memantine in behaviors and synaptic plasticity of a depression rat model. Neuroscience 2011; 182:88-97.
(36) Abolfazli R, Hosseini M, Ghanizadeh A, Ghaleiha A, Tabrizi M, Raznahan M et al. Double-blind randomized parallel-group clinical trial of efficacy of the combination fluoxetine plus modafinil versus fluoxetine plus placebo in the treatment of major depression. Depress Anxiety 2011; 28(4):297-302.
(37) Landriscina M, Bagala C, Piscazzi A, Schinzari G, Quirino M, Fabiano A et al. Nevirapine restores androgen signaling in hormone-refractory human prostate carcinoma cells both in vitro and in vivo. Prostate 2009; 69(7):744-54.
(38) Landriscina M, Altamura S A, Roca L, Gigante M, Piscazzi A, Cavalcanti E et al. Reverse transcriptase inhibitors induce cell differentiation and enhance the immunogenic phenotype in human renal clear-cell carcinoma. Int J Cancer 2008; 122(12):2842-50.
(39) Collins R, Armitage J, Parish S, Sleight P, Peto R. Effects of cholesterol-lowering with simvastatin on stroke and other major vascular events in 20536 people with cerebrovascular disease or other high-risk conditions. Lancet 2004; 363(9411):757-67.
(40) White C M, Greene L. Summary of AHRQ's Comparative Effectiveness Review of Angiotensin-Converting Enzyme Inhibitors or Angiotensin II Receptor Blockers Added to Standard Medical Therapy for Treating Stable Ischemic Heart Disease. J Manag Care Pharm 2011; 17(5):1-16.
(41) Gottlieb S S, Weinberg M. Cardiodepressant effects of mexiletine in patients with severe left ventricular dysfunction. Eur Heart J 1992; 13(1):22-7.
(42) Kammerer M, Brawek B, Freiman T M, Jackisch R, Feuerstein T J. Effects of antiepileptic drugs on glutamate release from rat and human neocortical synaptosomes. Naunyn Schmiedebergs Arch Pharmacol 2011; 383(5):531-42.
(43) Mark Hall. The WEKA Data Mining Software: An Update; SIGKDD Explorations. 11[1]. Jan. 1, 2009.
(44) Smoot M E, Ono K, Ruscheinski J, Wang P L, Ideker T. Cytoscape 2.8: new features for data integration and network visualization. Bioinformatics 2011; 27(3):431-2.
(45) Compton M T, Miller A H. Priapism associated with conventional and atypical antipsychotic medications: a review. J Clin Psychiatry 2001; 62(5):362-6.
(46) Iglesias G C, Santamarina M S, Alonso Villa M J. [Ziprasidone as coadjuvant treatment in resistant obsessive-compulsive disorder treatment]. Actas Esp Psiquiatr 2006; 34(4):277-9.
(47) Alexander J. De novo induction of obsessive-compulsive symptoms with quetiapine in a patient with borderline personality disorder. Aust N Z J Psychiatry 2009; 43(12):1185.
(48) Vulink N C, Denys D, Fluitman S B, Meinardi J C, Westenberg H G. Quetiapine augments the effect of citalopram in non-refractory obsessive-compulsive disorder: a randomized, double-blind, placebo-controlled study of 76 patients. J Clin Psychiatry 2009; 70(7):1001-8.
(49) Savas H A, Yumru M, Ozen M E. Quetiapine and ziprasidone as adjuncts in treatment-resistant obsessive-compulsive disorder: a retrospective comparative study. Clin Drug Investig 2008; 28(7):439-42.
(50) McMeekin H. Successful treatment of obsessive compulsive disorder with oxcarbazepine. A case report. J S C Med Assoc 2002; 98(8):316-20.
(51) van N L, de H L, van B N, Laan W, van den Brink W, Linszen D. Obsessive-compulsive symptoms in a randomized, double-blind study with olanzapine or risperidone in young patients with early psychosis. J Clin Psychopharmacol 2008; 28(2):214-8.
(52) Nelson E C. An open-label study of nefazodone in the treatment of depression with and without comorbid obsessive compulsive disorder Ann Clin Psychiatry 1994; 6(4):249-53.
(53) Peters B, de H L. Remission of schizophrenia psychosis and strong reduction of obsessive-compulsive disorder after adding clozapine to aripiprazole. Prog Neuropsychopharmacol Biol Psychiatry 2009; 33(8):1576-7.
(54) Schultheiss D, Muller S V, Nager W, Stief C G, Schlote N, Jonas U et al. Central effects of sildenafil (Viagra) on auditory selective attention and verbal recognition memory in humans: a study with event-related brain potentials. World J Urol 2001; 19(1):46-50.
(55) Umathe S N, Bhutada P S, Jain N S, Mundhada Y R, Borkar S S, Dhumal B. Role of nitric oxide in obsessive-compulsive behavior and its involvement in the anti-compulsive effect of paroxetine in mice. Nitric Oxide 2009; 21(2):140-7.
(56) Ghofrani H A, Osterloh I H, Grimminger F. Sildenafil: from angina to erectile dysfunction to pulmonary hypertension and beyond. Nat Rev Drug Discov 2006; 5(8):689-702.
(57) Ekins S, Andreyev S, Ryabov A, Kirillov E, Rakhmatulin E A, Bugrim A et al. Computational prediction of human drug metabolism. Expert Opin Drug Metab Toxicol 2005; 1(2):303-24.
(58) Nidhi, Glick M, Davies J W, Jenkins J L. Prediction of biological targets for compounds using multiple-category Bayesian models trained on chemogenomics databases. J Chem Inf Model 2006; 46(3):1124-33.
(59) Ong C S, Cook N, Lee S. Drug-related pemphigus and angiotensin converting enzyme inhibitors. Australas J Dermatol 2000; 41(4):242-6.
(60) Feleke E, Lyngstam O, Rastam L, Ryden L. Complaints of cold extremities among patients on antihypertensive treatment. Acta Med Scand 1983; 213(5):381-5.
(61) Hansen N T, Brunak S, Altman R B. Generating genome-scale candidate gene lists for pharmacogenomics. Clin Pharmacol Ther 2009; 86(2):183-9.
(62) Nidhi, Glick M, Davies J W, Jenkins J L. Prediction of biological targets for compounds using multiple-category Bayesian models trained on chemogenomics databases. J Chem Inf Model 2006; 46(3):1124-33.

1. A method for selecting a new therapeutic indication for at least one first pharmaceutical comprising the steps of:
a. generating a drug-side effect (SE) association for said at least one first pharmaceutical;
b. generating a disease-side effect (SE) association for at least one disease or disorder and at least one second pharmaceutical intended for treatment of said at least one disease or disorder;
c. determining an association strength between the drug-side effect (SE) association and the disease-side effect (SE) association;
d. selecting said at least one disease or disorder of (b) as a new therapeutic indication for said at least one first pharmaceutical if said at least one first pharmaceutical induces at least one side effect which is the same as at least one side effect induced by at least one second pharmaceutical intended for treatment of said at least one disease or disorder of (b).

2. The method of claim 1, wherein (a) includes counting the number of drugs inducing or not inducing a SE when treating or not treating a disease, and generating a confusion matrix.

3. The method of claim 1, wherein said drug-side effect (SE) association is determined by the pharmaceutical label of said first pharmaceutical

4. The method of claim 1, wherein said drug-side effect (SE) association is determined by the SIDER database.

5. The method of claim 1, wherein the association strength of (c) is determined using Matthew correlation coefficient (MCC).

6. The method of claim 1, wherein the association strength of (c) is determined using sensitivity (sn).

7. The method of claim 1, wherein the association strength of (c) is determined using specificity (sp).

8. A method of treating a mammal in need of treatment with at least one of the diseases listed as New Indication with the corresponding pharmaceutical listed as Drug:

| Drug | New Indication |
|---|---|
| acebutolol | Cardiomyopathy, Dilated |
| acebutolol | Hypercholesterolemia |
| allopurinol | Carotid Artery Diseases |
| allopurinol | Depressive Disorder |
| alprazolam | Depressive Disorder, Major |
| alprazolam | Depression, Postpartum |
| alprazolam | Obsessive-Compulsive Disorder |
| amiodarone | Carotid Artery Diseases |
| anastrozole | Lung Neoplasms |
| atorvastatin | Obsessive-Compulsive Disorder |
| atorvastatin | Depression, Postpartum |
| atorvastatin | Parkinson Disease |
| azathioprine | Lung Neoplasms |
| bezafibrate | Obsessive-Compulsive Disorder |
| bortezomib | Depression |
| bortezomib | Depressive Disorder, Major |
| bupropion | Cardiomyopathy, Hypertrophic |
| bupropion | Parkinson Disease |
| busulfan | Diabetes Mellitus |
| cabergoline | Depression |
| cabergoline | Depression, Postpartum |
| cabergoline | Depressive Disorder, Major |
| cabergoline | Lung Neoplasms |
| cabergoline | Ovarian Neoplasms |
| candesartan | Carotid Artery Diseases |
| candesartan | Depressive Disorder |
| capecitabine | Carotid Artery Diseases |
| capecitabine | Hypercholesterolemia |
| capecitabine | Hyperlipidemias |
| carbamazepine | Depression, Postpartum |
| carbamazepine | Obsessive-Compulsive Disorder |
| carvedilol | Lung Neoplasms |
| ceftriaxone | Depressive Disorder |
| celecoxib | Osteoporosis |
| celecoxib | Osteoporosis, Postmenopausal |
| cerivastatin | Cardiomyopathy, Hypertrophic |
| cerivastatin | Stroke |
| chlorambucil | Depression |
| chlorpromazine | Obsessive-Compulsive Disorder |
| cilazapril | Depressive Disorder |
| cilazapril | Obsessive-Compulsive Disorder |
| cisapride | Anxiety Disorders |
| cisapride | Depression, Postpartum |
| cisapride | Depressive Disorder, Major |
| cisapride | Obsessive-Compulsive Disorder |
| clomipramine | Carotid Artery Diseases |
| clomipramine | Epilepsy |
| clomipramine | Hypercholesterolemia |
| clomipramine | Hyperlipidemias |
| clomipramine | Osteoporosis |
| clomipramine | Osteoporosis, Postmenopausal |
| clomipramine | Parkinson Disease |
| clonidine | Depression, Postpartum |
| clopidogrel | Lung Neoplasms |
| clozapine | Cardiomyopathy, Dilated |
| clozapine | Cardiomyopathy, Hypertrophic |
| clozapine | Depression, Postpartum |
| clozapine | Depressive Disorder, Major |
| clozapine | Epilepsy |
| cyclophosphamide | Carotid Artery Diseases |
| cyclosporine | Depression, Postpartum |
| cyclosporine | Depressive Disorder |
| cyclosporine | Depressive Disorder, Major |
| cyclosporine | Obsessive-Compulsive Disorder |
| desipramine | Depression, Postpartum |
| desipramine | Cardiomyopathy, Hypertrophic |
| dexamethasone | Depression, Postpartum |
| dipyridamole | Depressive Disorder |
| dipyridamole | Obsessive-Compulsive Disorder |
| donepezil | Depression, Postpartum |
| donepezil | Obsessive-Compulsive Disorder |
| doxazosin | Obsessive-Compulsive Disorder |
| doxazosin | Carotid Artery Diseases |
| doxazosin | Depressive Disorder |
| doxazosin | Parkinson Disease |
| doxazosin | Schizophrenia |
| doxepin | Parkinson Disease |
| doxepin | Schizophrenia |
| doxorubicin | Epilepsy |
| doxorubicin | Obsessive-Compulsive Disorder |
| droperidol | Cardiomyopathy, Hypertrophic |
| duloxetine | Obsessive-Compulsive Disorder |
| duloxetine | Depression, Postpartum |
| duloxetine | Cardiomyopathy, Hypertrophic |
| efavirenz | Depression, Postpartum |
| efavirenz | Carotid Artery Diseases |
| efavirenz | Depressive Disorder, Major |
| eletriptan | Obsessive-Compulsive Disorder |
| eletriptan | Depressive Disorder |
| eletriptan | Carotid Artery Diseases |
| estradiol | Cardiomyopathy, Hypertrophic |
| exemestane | Ovarian Neoplasms |
| famotidine | Depressive Disorder, Major |
| famotidine | Lung Neoplasms |
| fenofibrate | Obsessive-Compulsive Disorder |
| flecainide | Epilepsy |
| fludarabine | Pain |
| fluoxetine | Carotid Artery Diseases |
| fluoxetine | Osteoporosis |
| fluoxetine | Osteoporosis, Postmenopausal |
| fluvastatin | Cardiomyopathy, Dilated |
| fluvastatin | Cardiomyopathy, Hypertrophic |
| fluvoxamine | Parkinson Disease |
| fluvoxamine | Cardiomyopathy, Hypertrophic |
| fluvoxamine | Carotid Artery Diseases |
| fluvoxamine | Diabetes Mellitus |
| formoterol | Depressive Disorder |
| formoterol | Obsessive-Compulsive Disorder |
| fosinopril | Depressive Disorder |
| fosinopril | Obsessive-Compulsive Disorder |
| gabapentin | Carotid Artery Diseases |
| gabapentin | Depression, Postpartum |
| gabapentin | Hypercholesterolemia |
| gabapentin | Hyperlipidemias |
| gadodiamide | Depressive Disorder, Major |
| ganciclovir | Hypercholesterolemia |
| ganciclovir | Cardiomyopathy, Dilated |
| ganciclovir | Cardiomyopathy, Hypertrophic |
| ganciclovir | Carotid Artery Diseases |
| ganciclovir | Hyperlipidemias |
| glatiramer acetate | Obsessive-Compulsive Disorder |
| glatiramer acetate | Depressive Disorder |
| glatiramer acetate | Depression, Postpartum |
| glatiramer acetate | Breast Neoplasms |
| glatiramer acetate | Carotid Artery Diseases |
| glatiramer acetate | Depression |
| glatiramer acetate | Depressive Disorder, Major |
| glatiramer acetate | Epilepsy |
| glatiramer acetate | Parkinson Disease |
| glatiramer acetate | Schizophrenia |
| glibenclamide | Depression |
| glimepiride | Depression |
| glipizide | Depression |
| granisetron | Depressive Disorder, Major |
| heparin | Obsessive-Compulsive Disorder |
| heparin | Parkinson Disease |
| hydrochlorothiazide | Anxiety Disorders |
| hydrochlorothiazide | Depressive Disorder, Major |
| hydrochlorothiazide | Obsessive-Compulsive Disorder |
| hydroxyurea | Carotid Artery Diseases |
| ifosfamide | Depression |
| imatinib | Cardiomyopathy, Hypertrophic |
| imatinib | Depression |
| imatinib | Depression, Postpartum |
| imatinib | Depressive Disorder |
| imipramine | Depression, Postpartum |
| ketorolac | Obsessive-Compulsive Disorder |
| lamivudine | Carotid Artery Diseases |
| lamotrigine | Obsessive-Compulsive Disorder |
| lamotrigine | Depression, Postpartum |
| lamotrigine | Osteoporosis |
| lamotrigine | Osteoporosis, Postmenopausal |
| lansoprazole | Depressive Disorder |
| lansoprazole | Obsessive-Compulsive Disorder |
| lansoprazole | Depression |
| lansoprazole | Depression, Postpartum |
| latanoprost | Osteoporosis |
| latanoprost | Osteoporosis, Postmenopausal |
| leflunomide | Lung Neoplasms |
| leflunomide | Obsessive-Compulsive Disorder |
| lenalidomide | Cardiomyopathy, Hypertrophic |
| lenalidomide | Breast Neoplasms |
| lenalidomide | Lung Neoplasms |
| levetiracetam | Depressive Disorder, Major |
| levonorgestrel | Anxiety Disorders |
| levonorgestrel | Depression |
| levonorgestrel | Depression, Postpartum |
| levonorgestrel | Depressive Disorder |
| levonorgestrel | Depressive Disorder, Major |
| lidocaine | Depression, Postpartum |
| lidocaine | Depressive Disorder |
| lidocaine | Depressive Disorder, Major |
| lidocaine | Obsessive-Compulsive Disorder |
| lithium | Depression, Postpartum |
| lovastatin | Cardiomyopathy, Hypertrophic |
| loxapine | Depression, Postpartum |
| loxapine | Depressive Disorder, Major |
| loxapine | Obsessive-Compulsive Disorder |
| memantine | Depression, Postpartum |
| memantine | Obsessive-Compulsive Disorder |
| methamphetamine | Cardiomyopathy, Hypertrophic |
| methyldopa | Anxiety Disorders |
| methyldopa | Depression, Postpartum |
| methyldopa | Depressive Disorder, Major |
| methyldopa | Obsessive-Compulsive Disorder |
| methylphenidate | Cardiomyopathy, Hypertrophic |
| mexiletine | Cardiomyopathy, Hypertrophic |
| mexiletine | Hypercholesterolemia |
| mexiletine | Stroke |
| mifepristone | Anxiety Disorders |
| mifepristone | Depression, Postpartum |
| mifepristone | Obsessive-Compulsive Disorder |
| modafinil | Depression, Postpartum |
| modafinil | Obsessive-Compulsive Disorder |
| morphine | Epilepsy |
| naproxen | Obsessive-Compulsive Disorder |
| naproxen | Depressive Disorder |
| naproxen | Osteoporosis |
| naproxen | Osteoporosis, Postmenopausal |
| nefazodone | Depression, Postpartum |
| nelfinavir | Carotid Artery Diseases |
| nevirapine | Breast Neoplasms |
| nevirapine | Lung Neoplasms |
| nevirapine | Ovarian Neoplasms |
| olanzapine | Depression, Postpartum |
| olanzapine | Carotid Artery Diseases |
| olanzapine | Epilepsy |
| omeprazole | Carotid Artery Diseases |
| omeprazole | Depressive Disorder |
| omeprazole | Hypercholesterolemia |
| omeprazole | Hyperlipidemias |
| omeprazole | Obsessive-Compulsive Disorder |
| ondansetron | Depressive Disorder, Major |
| oseltamivir | Depression |
| oseltamivir | Depression, Postpartum |
| oseltamivir | Depressive Disorder, Major |
| oxaliplatin | Obsessive-Compulsive Disorder |
| oxcarbazepine | Obsessive-Compulsive Disorder |
| oxcarbazepine | Depression, Postpartum |
| oxcarbazepine | Depressive Disorder, Major |
| oxcarbazepine | Parkinson Disease |
| oxycodone | Depression |
| paclitaxel | Depressive Disorder, Major |
| pamidronate | Lung Neoplasms |
| paroxetine | Carotid Artery Diseases |
| pemoline | Epilepsy |
| penicillin g | Obsessive-Compulsive Disorder |
| penicillin g | Schizophrenia |
| pergolide | Obsessive-Compulsive Disorder |
| pergolide | Depression, Postpartum |
| pergolide | Schizophrenia |
| perindopril | Cardiomyopathy, Hypertrophic |
| phenytoin | Carotid Artery Diseases |
| phenytoin | Depression, Postpartum |
| phenytoin | Obsessive-Compulsive Disorder |
| pindolol | Hypercholesterolemia |
| posaconazole | Lung Neoplasms |
| pramipexole | Obsessive-Compulsive Disorder |
| pramipexole | Depression, Postpartum |
| pramipexole | Breast Neoplasms |
| pramipexole | Carotid Artery Diseases |
| pramipexole | Epilepsy |
| pravastatin | Cardiomyopathy, Dilated |
| pravastatin | Cardiomyopathy, Hypertrophic |
| prednisolone | Obsessive-Compulsive Disorder |
| prednisolone | Anxiety Disorders |
| prednisolone | Depression, Postpartum |
| prednisone | Anxiety Disorders |
| prednisone | Carotid Artery Diseases |
| prednisone | Depression, Postpartum |
| prednisone | Depressive Disorder, Major |
| prednisone | Obsessive-Compulsive Disorder |
| procarbazine | Depressive Disorder |
| procarbazine | Epilepsy |
| propoxyphene | Carotid Artery Diseases |
| propylthiouracil | Lung Neoplasms |
| quetiapine | Depression, Postpartum |
| rabeprazole | Depressive Disorder |
| rabeprazole | Depression, Postpartum |
| rabeprazole | Carotid Artery Diseases |
| rabeprazole | Depression |
| rabeprazole | Depressive Disorder, Major |
| rabeprazole | Hypercholesterolemia |
| rabeprazole | Hyperlipidemias |
| rabeprazole | Lung Neoplasms |
| rabeprazole | Obsessive-Compulsive Disorder |
| reserpine | Osteoporosis |
| reserpine | Osteoporosis, Postmenopausal |
| ribavirin | Obsessive-Compulsive Disorder |
| risperidone | Depression, Postpartum |
| ritonavir | Depressive Disorder |
| ritonavir | Depressive Disorder, Major |
| ritonavir | Lung Neoplasms |
| ritonavir | Obsessive-Compulsive Disorder |
| ritonavir | Osteoporosis, Postmenopausal |
| rivastigmine | Obsessive-Compulsive Disorder |
| rivastigmine | Depression, Postpartum |
| rivastigmine | Depressive Disorder, Major |
| rivastigmine | Epilepsy |
| rofecoxib | Carotid Artery Diseases |
| rosuvastatin | Cardiomyopathy, Dilated |
| rosuvastatin | Cardiomyopathy, Hypertrophic |
| saquinavir | Carotid Artery Diseases |
| saquinavir | Depression |
| saquinavir | Depression, Postpartum |
| saquinavir | Depressive Disorder |
| sibutramine | Obsessive-Compulsive Disorder |
| sildenafil | Obsessive-Compulsive Disorder |
| sildenafil | Depression, Postpartum |
| simvastatin | Cardiomyopathy, Hypertrophic |
| sotalol | Hypercholesterolemia |
| sulfasalazine | Cardiomyopathy, Hypertrophic |
| sumatriptan | Depression, Postpartum |
| sumatriptan | Depressive Disorder, Major |
| sumatriptan | Carotid Artery Diseases |
| sumatriptan | Epilepsy |
| sumatriptan | Parkinson Disease |
| tacrolimus | Epilepsy |
| tadalafil | Obsessive-Compulsive Disorder |
| tadalafil | Parkinson Disease |
| tadalafil | Schizophrenia |
| tamoxifen | Obsessive-Compulsive Disorder |
| tamoxifen | Parkinson Disease |
| tegaserod | Depressive Disorder |
| tegaserod | Obsessive-Compulsive Disorder |
| telmisartan | Obsessive-Compulsive Disorder |
| temozolomide | Carotid Artery Diseases |
| temozolomide | Depression, Postpartum |
| temozolomide | Depressive Disorder |
| temozolomide | Depressive Disorder, Major |
| temozolomide | Obsessive-Compulsive Disorder |
| testosterone | Obsessive-Compulsive Disorder |
| testosterone | Cardiomyopathy, Dilated |
| testosterone | Cardiomyopathy, Hypertrophic |
| thalidomide | Depression |
| thalidomide | Depressive Disorder |
| thalidomide | Depressive Disorder, Major |
| thalidomide | Anxiety Disorders |
| thalidomide | Cardiomyopathy, Hypertrophic |
| thalidomide | Depression, Postpartum |
| thioridazine | Obsessive-Compulsive Disorder |
| thioridazine | Depression, Postpartum |
| thioridazine | Depressive Disorder, Major |
| tiagabine | Depression, Postpartum |
| tiagabine | Obsessive-Compulsive Disorder |
| timolol | Cardiomyopathy, Hypertrophic |
| timolol | Cardiomyopathy, Dilated |
| timolol | Hypercholesterolemia |
| timolol | Obsessive-Compulsive Disorder |
| tolbutamide | Depression |
| tolcapone | Depression |
| tolcapone | Depression, Postpartum |
| tolcapone | Depressive Disorder, Major |
| tolcapone | Obsessive-Compulsive Disorder |
| topiramate | Depression, Postpartum |
| topiramate | Cardiomyopathy, Hypertrophic |
| tramadol | Depressive Disorder |
| tramadol | Carotid Artery Diseases |
| tramadol | Depression, Postpartum |
| tramadol | Depressive Disorder, Major |
| trazodone | Depression, Postpartum |
| triamcinolone | Anxiety Disorders |
| triamcinolone | Carotid Artery Diseases |
| triamcinolone | Depression, Postpartum |
| triamcinolone | Depressive Disorder |
| triamcinolone | Depressive Disorder, Major |
| triamcinolone | Obsessive-Compulsive Disorder |
| trifluoperazine | Depressive Disorder, Major |
| trifluoperazine | Obsessive-Compulsive Disorder |
| trifluoperazine | Depression |
| trifluoperazine | Depression, Postpartum |
| trifluoperazine | Depressive Disorder |
| trimipramine | Anxiety Disorders |
| trovafloxacin | Depression, Postpartum |
| trovafloxacin | Depressive Disorder |
| trovafloxacin | Depressive Disorder, Major |
| trovafloxacin | Obsessive-Compulsive Disorder |
| venlafaxine | Osteoporosis |
| venlafaxine | Osteoporosis, Postmenopausal |
| venlafaxine | Parkinson Disease |
| venlafaxine | Carotid Artery Diseases |
| verapamil | Depressive Disorder, Major |
| verapamil | Depression, Postpartum |
| verapamil | Obsessive-Compulsive Disorder |
| vincristine | Cardiomyopathy, Hypertrophic |
| vincristine | Epilepsy |
| vincristine | Obsessive-Compulsive Disorder |
| vinorelbine | Depression |
| warfarin | Obsessive-Compulsive Disorder |
| warfarin | Parkinson Disease |
| zanamivir | Depression |
| zanamivir | Depression, Postpartum |
| zanamivir | Depressive Disorder, Major |
| ziprasidone | Obsessive-Compulsive Disorder |
| ziprasidone | Depression, Postpartum |
| ziprasidone | Breast Neoplasms |
| ziprasidone | Carotid Artery Diseases |
| ziprasidone | Epilepsy |
| zolpidem | Depression, Postpartum |
| zolpidem | Obsessive-Compulsive Disorder |
| zolpidem | Osteoporosis |
| zolpidem | Osteoporosis, Postmenopausal |
| zonisamide | Obsessive-Compulsive Disorder |
